(19) United States
(12) Patent Application Publication
(10) Pub. No.: US 2005/0234107 A1
(43) Pub. Date: Oct. 20, 2005

(54) Use of diuretics for treating swellings

(76) Inventor: Rudolf Wank, Munchen (DE)

Correspondence Address: Burns Doane Swecker & Mathis, PO Box 1404, Alexandria, VA, 22313-1404, US

(21) Appl. No.: 10/516,018

(22) PCT Filed: May 27, 2003
(86) PCT No.: PCT/EP03/05575

§ 371 Date: Nov. 29, 2004

(30) Foreign Application Priority Data
May 29, 2002 (DE) 102 23 932.0

(51) Int. Cl. A61K 31/34; A61K 31/44

(52) U.S. Cl. 514/345; 514/471

(57) ABSTRACT

The present invention relates to the use of a diuretic, especially a loop diuretic, for the treatment of local skin swellings. The invention further relates to pharmaceutical preparations of diuretics in topical form.

The present invention relates to topical preparations containing diuretics, especially loop diuretics, and to their use for the treatment of local skin swellings.

Diuretics are understood as meaning substances which cause an increased excretion of water and salt. Their field of use covers both the treatment of kidney diseases and the treatment of arterial hypertonia, cardiac insufficiency and the mobilization of general or organic oedemas. Diuretics are subdivided into different groups according to their principle of action, said groups including osmotic diuretics, carboanhydrase inhibitors, thiazide diuretics, loop diuretics, potassium sparing diuretics and aldosterone antagonists.

Loop diuretics are used particularly in the treatment of organic oedemas (especially of cardiac, renal or hepatic origin) and as antihypertensives. They are characterized by an inhibitory effect on the reabsorption of chloride and sodium in the ascending limb of Henle's loop and are also effective in cases of reduced glomerular filtration rate. One of the most prominent representatives of loop diuretics is furosemide, the group also including azosemide, bumetanide, piretanide and torasemide. Loop diuretics are administered orally or intravenously and are used in both short-term and long-term therapies. EP 0 247 507 describes a transdermic therapeutic system for loop diuretics which produces a retarded systemic action via percutaneous absorption.

It has now been found, surprisingly, that topical pharmaceutical preparations of diuretics, especially loop diuretics, are suitable for the local treatment of skin swellings. Their effect can even markedly exceed that of the intravenous infusion of loop diuretics, or they can facilitate the action of previously inactive intravenous infusions of loop diuretics. This makes it possible to treat local oedemas which previously could only be treated inadequately by manual drainage.

The invention therefore relates to the use of a diuretic, especially a loop diuretic, or a pharmaceutically acceptable salt thereof for the manufacture of a topical pharmaceutical preparation for the treatment of local skin swellings, and to the topical preparations themselves.

Within the framework of the present invention, skin swellings include particularly oedematous swellings or swellings caused by a venous or lymphatic inflammation.

Within the framework of the present invention, it is possible in principle to use any of the types of diuretic mentioned above. These include e.g. thiazide diuretics, such as hydrochlorothiazide (6-chloro-3,4-dihydro-2H-1,2,4-benzothiadiazine-7-sulfonamide 1,1-dioxide) and ethacrynic acid ([2,3-dichloro-4-(2-ethylacryloyl)phenoxy]acetic acid), carboanhydrase inhibitors, such as acetazolamide (5-acetylamino-1,3,4-thiadiazole-2-sulfonamide), potassium sparing diuretics, such as triamterene (2,4,7-triamino-6-phenylpteridine), and especially loop diuretics.

The loop diuretics which can be used within the framework of the present invention include furosemide (4-chloro-N-furfuryl-5-sulfamoylanthranilic acid), azosemide (2-chloro-5-(1H-tetrazol-5-yl)-2-thienylsulfanilamide), bumetanide (3-butylamino-4-phenoxy-5-sulfamoylbenzoic acid), piretanide (4-phenoxy-3-(1-pyrrolidinyl)-5-sulfamoylbenzoic acid) and torasemide (1-isopropyl-3-[(4-toluidino-3-pyridyl)sulfonyl]urea). It is preferable to use furosemide, piretanide and torasemide and very particularly preferable to use furosemide. Pharmaceutical preparations of these active substances in tablet form or as substances in tablet form or as injection solutions are commercially available.

Possible pharmaceutically acceptable salts are both water-soluble and sparingly water-soluble salts that are generally known to those skilled in the art. The topical preparations according to the invention, which contain one or more diuretics, especially loop diuretics, as active substances and are suitable for application to the skin, include ointments, pastes, lotions, creams, gels and tinctures. Within the framework of the present invention, tinctures include liquid preparations containing active substances which are suitable for application to the skin but are not intended for oral or intravenous administration.

The preparations are manufactured in known manner using the known and conventional pharmaceutical auxiliary substances and other conventional excipients and diluents (List et al., Arzneiformenlehre, Wiss. Verlagsges., Stuttgart, 1985; Sucka et al., Pharmazeutische Technologie, Thieme Verlag, Stuttgart, 1978).

In the manufacture of an ointment, for example, it is possible to use organogels (e.g. Vaseline, Plastibase), lipogels (e.g. beeswax), hydrogels (e.g. Aerosil®, bentonite, starch derivatives, polyacrylic acid, polyethylene glycols) or silicone gels as the ointment base. A preferred ointment base is Vaseline, it also being possible for the ointment to contain other materials such as fatty alcohols, glyceryl monostearates, triglycerides, alkylene glycols, dimethyl sulfoxide and water. A very particularly preferred ointment base is the DAC base cream (Cremor basalis, Deutscher Arzneimittel-Codex), which contains white Vaseline, glycerol monostearate 60, cetyl alcohol, medium-chain triglycerides, macrogol 1000 glycerol monostearate, propylene glycol and water.

Creams can be manufactured using the above-mentioned ointment bases in combination with larger amounts of water and also, in particular, fats and oils, waxes, fatty acids and fatty acid esters, long-chain alcohols and emulsifiers.

The preparations in gel form include organogels (i.e. hydrocarbon gels, e.g. Vaseline, Plastibase), lipogels (e.g. natural fats, beeswax), hydrogels (e.g. hydroxypropyl cellulose, hydroxypropyl methyl cellulose, Aerosil®, bentonite, starch derivatives, polyacrylic acid, polyethylene glycols), silicone gels or emulsion gels containing conventional emulsifiers, as well as oleogels composed essentially of liquid paraffin with polyethylene or oils and thickeners.

It is further possible to add preservatives, stabilizers, buffer substances, colourants, antioxidants, etc.

Tinctures for topical application to the skin can be manufactured using e.g. water or physiologically acceptable solvents like alcohols (ethanol, propylene glycol or polyglycols), oils or paraffins.

The pharmaceutical topical preparations according to the invention contain the diuretic or a pharmaceutically acceptable salt thereof in an amount of 0.1 to 10 wt. %, based on the pharmaceutical preparation. The active substance content is preferably in the range from 0.1 to 1 wt. % and very particularly preferably 0.2 wt. %.

To introduce the diuretics into the topical preparations according to the invention, it is possible to use commercially available active substance formulations, for example solutions in ampoule form. Various furosemide solutions are obtainable e.g. from Hoechst (Lasix®), CT (Furo®), Ratiopharm (Furosemid-Ratiopharm®) and Hexal (Furorese®).

In a preferred embodiment, the pharmaceutical preparation is in the form of an ointment containing furosemide or a pharmaceutically acceptable salt thereof in an amount of 0.2 wt. %, based on the pharmaceutical preparation.

Without intentionally implying a restriction to one particular theory in order to explain the ascertainable action of diuretics, especially loop diuretics, the mechanisms of action illustrated below are considered.

Obstruction of the venous and lymphatic discharge, and the resulting reflux in both vascular systems, are described as major factors of the local oedema.

Loop diuretics inhibit Na+K+Cl cotransporters. There are at least six different isoforms of absorbing cotransporters. A cotransporter, namely a secretory cotransporter, was first isolated from an anal gland of the shark. Whereas the “absorbing” cotransporter seems to be expressed only in the kidney, secretory cotransporters are expressed in many tissues. The local effect of furosemide could therefore involve a blockade of a secretory cotransporter.

Recently, however, many results have come to light which have qualified various tissues as having a “single” specialized function. It has been shown that immune cells produce neurotrophins and pituitary hormones whose production had long been ascribed only to brain cells. An invariant chain of the T cell receptor has recently been discovered on brain cells. Thus it seems that a receptor or a gene product is first discovered in the tissue with the highest expression, i.e. at the site of most intense utilization, but other tissues are capable of performing the same tasks at a lower level with the same molecules.

However, yet another important effect of loop diuretics is known which cannot be explained by the “classic” accumulation at the Cl binding site of the cotransporter protein in the ascending limb of Henle's loop:

Loop diuretics increase the venous absorption capacity (vasodilatory effect). The mode of action is not known. The profound action of loop diuretics on pulmonary oedema starts before diuresis and is attributed to the increase in venous absorption capacity.

Thus the local mode of action could be connected with an inhibition of the secretory cotransporter and/or a vasodilatory effect. However, loop diuretics are also capable of inhibiting Na+K+ATPase, glycolysis, mitochondrial respiration, the microsomal Ca²⁺ pump, adenylcyclase, phosphodiesterase and prostaglandin dehydrogenase. These inhibitory effects on electrolyte transport, previously observed only in vitro, could be observed in many tissues only at high concentrations. The application of an ointment might be capable of achieving such high concentrations locally.

The pharmaceutical preparation in topical form is applied to the affected areas of skin several times a day and as a rule twice to four times a day. The frequency of application can be adapted as required. The local treatment of a swelling with the preparation according to the invention can be administered together with a medicinal treatment appropriate for the disease.

The invention is illustrated in greater detail below with the aid of Examples.

### Preparation of a Furosemide Ointment

The DAC base cream (Cremor basalis, Deutsche Arzneimittel-Codex) having the following composition was used as the ointment base:

per 100 g of formulation:

| | |
|---|---|
| Glycerol monostearate 60 | 4.0 g |
| Cetyl alcohol | 6.0 g |
| Medium-chain triglycerides | 7.5 g |
| White Vaseline | 25.5 g |
| Macrogol 1000 glycerol monostearate | 7.0 g |
| Propylene glycol | 10.0 g |
| Water | 40 g |

The ointment was prepared using 68 g of DAC base cream, 12 g of dimethyl sulfoxide and 200 mg of furosemide (20 ml of Lasix®, active substance concentration 10 mg/ml).

### Patient Studies

The local application of furosemide ointment (from the above Example) on a cancer patient with swelling of the genitals after leukocyte apheresis led to a marked regression of the swelling within a few hours. This effect could not be achieved by a previous intravenous administration of furosemide in combination with spironolactone, which is clear evidence of the local action of furosemide ointment. Furosemide in ointment form is only effective locally, not systemically, with the result that there is no interference with the patients' electrolyte metabolism.

Treatment of a so-called lymph arm in a breast cancer patient with furosemide ointment (from the above Example) led to a marked reduction in the oedema after a few applications, making it unnecessary to carry out a lymph drainage.

In another female patient, an armpit swelling attributable to a venous inflammation could be markedly reduced within 12 hours by the local application of furosemide ointment (from the above Example).

The furosemide-containing ointment according to the invention led within a short time to a marked regression of swellings of very different types and thus opens up novel and efficient approaches to the treatment of swellings.

1-22. (canceled)

23. A pharmaceutical composition in topical form for the treatment of local skin swellings comprising an effective amount of a diuretic or pharmaceutically acceptable salt thereof and a pharmaceutically acceptable carrier therefore.

24. The pharmaceutical composition according to claim 23, wherein the diuretic is a loop diuretic.

25. The pharmaceutical composition according to claim 23, wherein the loop diuretic is furosemide, azosemide, bumetanide, piretanide or torasemide or a pharmaceutically acceptable salt thereof.

26. The pharmaceutical composition according to claim 25, wherein the loop diuretic is furosemide or a pharmaceutically acceptable salt thereof.

27. The pharmaceutical composition according to claim 23, wherein the pharmaceutical composition comprises a diuretic or a pharmaceutically acceptable salt thereof in an amount of 0.1 to 10 wt. %, based on the pharmaceutical preparation.

28. The pharmaceutical composition according to claim 27, wherein the pharmaceutical composition comprises a diuretic or a pharmaceutically acceptable salt thereof in an amount of 0.2 wt. %, based on the pharmaceutical preparation.

29. The pharmaceutical composition according to claim 23, wherein the pharmaceutical topical composition is in the form of an ointment, paste, lotion, cream, tincture or gel.

30. The pharmaceutical composition according to claim 23, wherein the pharmaceutical composition comprises conventional pharmaceutical auxiliary substances, excipients and/or diluents.

31. The pharmaceutical composition according to claim 23, wherein the pharmaceutical composition is applied locally to the affected areas of skin 2-4 times a day.

32. The pharmaceutical composition according to claim 23, wherein the pharmaceutical composition further comprises another medicinal treatment appropriate for the disease.

33. A method for the treatment of local skin swellings caused by venous or lymphatic inflammation comprising topically applying a diuretic or a pharmaceutically acceptable salt thereof to a patient in need of such treatment.

34. The method according to claim 33, wherein the diuretic is a loop diuretic.

35. The method according to claim 33, wherein the loop diuretic is furosemide, azosemide, bumetanide, piretanide or torasemide or a pharmaceutically acceptable salt thereof.

36. The method according to claim 35, wherein the loop diuretic is furosemide or a pharmaceutically acceptable salt thereof.

37. The method according to claim 33, wherein the pharmaceutical composition comprises a diuretic or a pharmaceutically acceptable salt thereof in an amount of 0.1 to 10 wt. %, based on the pharmaceutical preparation.

38. The method according to claim 37, wherein the pharmaceutical composition comprises a diuretic or a pharmaceutically acceptable salt thereof in an amount of 0.2 wt. %, based on the pharmaceutical preparation.

39. The method according to claim 33, wherein the pharmaceutical topical composition is in the form of an ointment, paste, lotion, cream, tincture or gel.

40. The method according to claim 33, wherein the pharmaceutical composition comprises conventional pharmaceutical auxiliary substances, excipients and/or diluents.

41. The method according to claim 33, wherein the pharmaceutical composition is applied locally to the affected areas of skin 2-4 times a day.

42. The method according to claim 33, wherein the pharmaceutical composition further comprises another medicinal treatment appropriate for the disease.
